(19) United States
(12) Patent Application Publication
(10) Pub. No.: US 2020/0222676 A1
(43) Pub. Date: Jul. 16, 2020

(54) PRESSURE-REGULATED BALLOON APPARATUS POSITIONED IN THE CAVITY FORMED IN THE BRAIN AFTER SURGERY

(71) Applicant: ATATURK UNIVERSITESI BILIMSEL ARASTIRMA PROJELERI BIRIMI, Erzurum (TR)

(72) Inventor: Mehmet Dumlu AYDIN, Erzurum (TR)

(21) Appl. No.: 16/628,763

(22) PCT Filed: Jan. 16, 2019
(86) PCT No.: PCT/TR2019/050033

§ 371 Date: Jan. 6, 2020

(30) Foreign Application Priority Data
Jan. 25, 2018 (TR) 2018/01062

(51) Int. Cl. A61M 29/02; A61B 17/02

(52) U.S. Cl. CPC A61M 29/02; A61B 2017/00557; A61B 17/0218

(57) ABSTRACT

A pressure-regulated balloon apparatus is provided to prevent brain collapse, to solve negative/positive pressure imbalances formed, and to prevent bleeding by pressure with a soft effect to the cavity formed after brain cyst and tumor surgery.

### TECHNICAL FIELD

The invention relates to a pressure-regulated balloon apparatus positioned in a cavity to prevent the negativities caused by the cavities formed in the brain after the brain and nerve surgery.

In particular, the invention relates to a pressure-regulated balloon apparatus used to prevent the brain collapse, to solve negative/positive pressure imbalances to be formed, and to prevent bleeding by pressure with a soft effect to the cavity formed after the brain cyst and tumor surgery.

### STATE OF THE ART

The brain diseases are various diseases such as bleedings, cysts, tumors, inflammations in the brain. The various diseases in the brain which is the most important organ major defects in another part of the body. The treatment options are determined by the physician according to the type of cyst and tumor, their location and the results of treatment in the literature. In general, however, the options are surgical treatment, radiation therapy (radiotherapy) and chemotherapy. In most of the brain cysts and tumors, surgical intervention is the prioritized treatment method.

After the giant cysts and tumors in the brain are emptied, the cortex may be folded, curved, its veins ruptured and rotten by collapsing into the cavity. These negativities can cause disability or death after surgery.

In the state of the art, the method of balloon-assisted hydatic cyst removal is used in the brain cysts⁽¹⁾. Said surgical method is applied during the standard surgical interventions such as hydatic cyst surgery or wide dura opening during the classical wide craniotomy. First, the Foley probe at the end of the inflated balloon is gradually progressed between the cyst and brain tissue. Then the brain tissue is cleaned from the hydatic cyst by inflating the balloon with the saline solution.

The balloon used in the present applications is used to remove the cyst from the brain tissue. No studies have been carried out related to the placement of the balloon and any devices such as balloon and the like in the cavity formed by the cyst and tumor removed from the brain tissue.

In conclusion, an improvement in the technical field has been required due to the aforementioned negativities and the inadequacy of the existing solutions related to the issue.

### THE AIM OF THE INVENTION

The present invention relates to a pressure-regulated balloon apparatus (Dadalon) positioned in the cavity formed in the brain after surgery which meets the aforementioned requirements and resolves all disadvantages and provides additional advantages.

The aim of the invention is to prevent the brain collapse into the cavity formed after the cyst and tumor surgery.

The aim of the invention is to solve the negative/positive pressure imbalances to be formed after the brain cyst and tumor surgery.

The aim of the invention is to prevent bleeding by pressure with a soft effect to the cavity formed after the brain cyst and tumor surgery.

The aim of the invention is to prevent the cortex from being folded, curved, rupture of the veins and rotten by collapsing into the cavity formed after the giant cysts and tumors in the brain are emptied.

It is an object of the invention to prevent the increased intracranial pressure caused by the expanding brain and to prevent the brain from remaining under pressure.

It is an object of the invention to provide expansion in the collapsed brain.

It is an object of the invention to prevent the complications such as increased pressure in the brain, bleeding, folding of the brain vessels and decreasing of flow, and stopping by spontaneous shrinking of the balloon remained under the increased pressure.

In order to perform the aforementioned aims, the invention is a pressure-regulated balloon apparatus positioned in a cavity to prevent the negativities caused by the cavities formed in the brain after the brain and nerve surgery, comprising:
a main balloon which provides a soft support to the brain with the air/water pumped into it,
a security balloon outside the skull,
a double-channel probe pumping air/water into the security balloon with said main balloon and draining the existing air/water,
a valve positioned on the probe to throw out the air/water in the main balloon by opening automatically when the brain pressure exceeds the normal.

The structural and characteristic features, and all advantages of the invention will be more clearly understood by the following detailed description, and therefore it is necessary to make the evaluation by considering this detailed description.

### BRIEF DESCRIPTION OF DRAWINGS

FIG. 1: is a general view of the pressure-regulated balloon apparatus (Dadalon) according to the invention.

FIG. 2: is a general view of the valve when the brain pressure is low.

FIG. 3: is a general view of the valve when the brain pressure increases.

FIG. 4: is a representative view of the pressure-regulated balloon (Dadalon) applied to the brain after the cyst and tumor are removed.

FIG. 5: is a representative view of the pressure-regulated balloon movement during the normalization process of the brain.

The drawings do not have to be scaled and the details which are not required to understand the present invention may be neglected. Moreover, the elements which are at least substantially identical or have at least substantially identical functions are indicated by the same number.

### DESCRIPTION OF THE PART REFERENCES

10. Pressure-regulated balloon apparatus
11. Main balloon
12. Neck
13. Security balloon
14. Probe
15. Valve
151. Piston
152. Spring
153. Output end
16. Reserve valve
B. Brain
K. Skull

### DETAILED DESCRIPTION OF THE INVENTION

In this detailed description, the pressure-regulated balloon apparatus (Dadalon) (10), positioned in said cavity to prevent the negativities caused by the cavities formed in the brain (B) after the brain and nerve surgery is merely described for a better understanding of the subject and without any limiting effects.

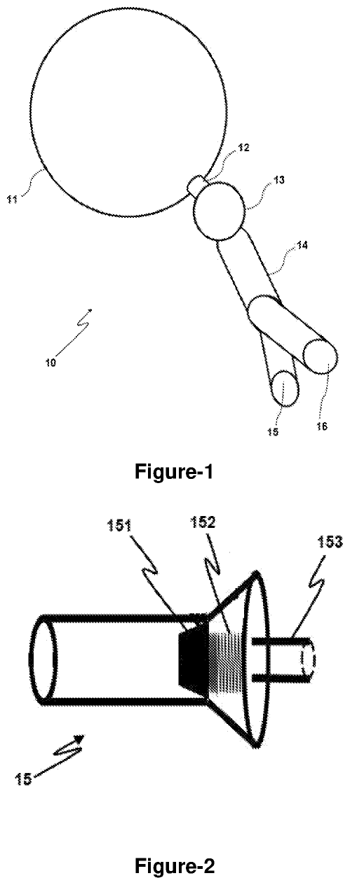

The pressure-regulated balloon apparatus (10) of the invention provided in FIG. 1, in terms of its general structure, consists of the main balloon (11) providing a soft support to the brain (B) with the air/water pumped into it, the security balloon (13) outside of the skull (K), the double-channel probe (14) pumping air/water into the security balloon with said main balloon (11) and draining the existing air/water, the valve (15) positioned on the probe to throw out the air/water in the main balloon (11) by opening automatically when the brain (B) pressure exceeds the normal.

The main balloon (11) of the pressure-regulated balloon apparatus (10) is a normal balloon which is positioned in the brain (B) and in which the probe (14) is inserted after being sterilized. Said probe (14) is the Foley probe. The main balloon (10) is inflated such that it fills the cavity formed in the brain (B) by means of air/water through the probe (14). Then, the security balloon (13) which is outside the skull (K), is also inflated by giving air/water with the probe (14). In FIG. 4, a representative view of the pressure-regulated balloon apparatus (Dadalon) (10) applied to the brain (B) after the removal of cyst and tumor is provided.

The neck (12) positioned between the main balloon (11) and the security balloon (13) is located between the brain membranes, bone and scalp to prevent the pressure-regulated balloon apparatus (Dadalon) (10) from sliding in and out.

In FIG. 2, when the brain pressure is low, the valve (15) whose general view is given, is pressure regulated, and comprises the piston (151) moving according to the pressure formed in the brain (B), the spring (152) moving the piston (151) forward (A direction) by compressing in case of the cylinder exceeds the brain pressure and preventing the output of the air/water by opening when the pressure decreases and the output end (153) from which the air/water is drained.

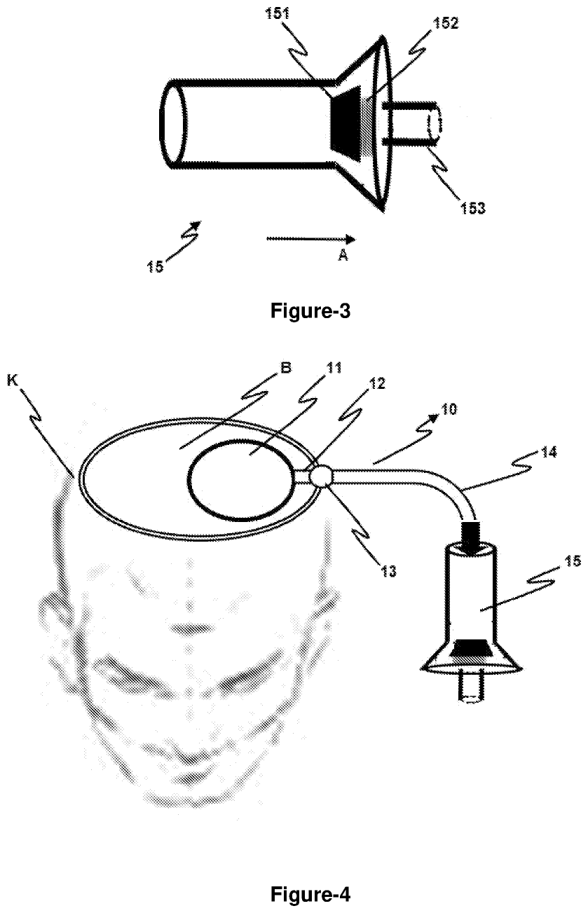

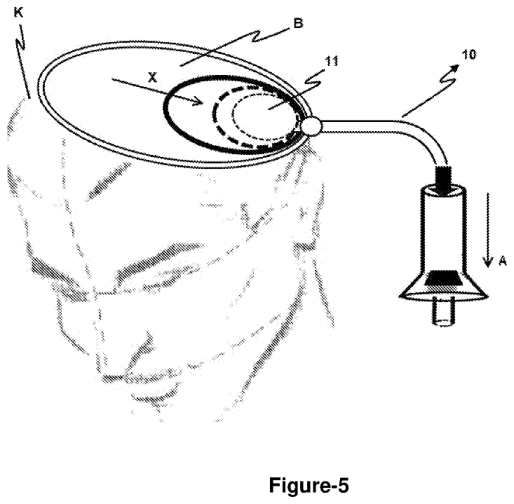

The valve (15) is pressure-regulated and the spring (152) is compressed when the pressure exceeds 15 cm of water where the pressure-regulated balloon apparatus (10) is the exit foramen of the pressure-regulated balloon apparatus (10) in the brain (B), and the piston (151) moves forward (A direction) to throw away the air/water in the main balloon (11) from the output end (153). Thus, in order to prevent the increase of intracranial pressure to be formed by the expanding brain and to prevent the brain (B) from being under pressure, the volume of the main balloon (11) is shrinking in the X direction and an expansion in the brain (B) is provided. The diameter of the main balloon (11) shrinking by itself in this way is removed when it is less than 2 cm in few days. In FIG. 3, the general view of the valve (15) when the brain pressure increases is provided. In FIG. 5, the representative view of the pressure-regulated balloon movement (10) during the normalization process of the brain (B).

There is a non-automated reserve valve (16) with 30 cm water pressure-regulated at the end of the valve (14), where the security balloon (13) has an exit foramen, opening while pushing the reserve air/water and then closing to prevent the reserve from being deflated. The reserve valve (16) is obtained by inverting the valve (15).

The application of the pressure-regulated balloon apparatus (Dadalon) (10) has provided very successful results in the use after the cysts and tumor surgeries. It prevents the intracranial pressure increase to be formed by the expanding brain (B) and the pressure of the brain (B) and provides expansion in the collapsed brain (B). The main balloon (11) which is under the increased pressure shrinks by itself in X direction and prevents the complications such as pressure increase in the brain (B), bleeding, folding of the brain vessels and decreasing of the flow and stopping.

The pressure-regulated balloon apparatus (10) of the invention can be used after the brain hemorrhage surgeries.

### REFERENCES

1) Ulutas M., Cinar K., Secer M., Removal of large hydatic cysts with balloon-assisted modification of Dowling's method: technical report. 2015, Acta Neurochir (Wien). 157(7): S1221-1224

1. A pressure-regulated balloon apparatus positioned in a cavity to prevent negativities caused by cavities formed in the brain after brain and nerve surgery, characterized by comprising:
a main balloon which provides a soft support to the brain with air/water pumped into it;
a security balloon outside the skull;
a double-channel probe pumping air/water into the security balloon with said main balloon and draining the existing air/water;
a valve positioned on the probe to throw out the air/water in the main balloon by opening automatically when the brain pressure exceeds the normal.

2. The pressure-regulated balloon apparatus according to claim 1, further comprising a neck positioned between the main balloon and the security balloon, remaining in between the cortex, bone and scalp to prevent the pressure-regulated balloon apparatus from sliding in and out.

3. The pressure-regulated balloon apparatus according to claim 1, wherein said valve comprises a piston that moves according to the pressure created in the brain, a spring that moves the piston forward by compressing in case the cylinder exceeds the brain pressure and that prevents the output of the air/water by opening when the pressure decreases and an output end from which the air/water is drained.

4. The pressure-regulated balloon apparatus according to claim 1, further comprising a reserve valve positioned at the end of the valve, opening while pushing the reserve air/water and then closing to prevent the reserve from being deflated.

5. The pressure-regulated balloon apparatus according to claim 1, wherein said reserve valve is obtained by inverting the valve.

6. The pressure-regulated balloon apparatus according to claim 1, wherein said probe is a Foley probe.
